(19) United States
(12) Patent Application Publication
(10) Pub. No.: US 2024/0141285 A1
(43) Pub. Date: May 2, 2024

(54) Artificial Sputum and Respiratory Epithelial Model for Bacterial Biofilms in the Respiratory Tract

(71) Applicant: University of Pittsburgh - Of the Commonwealth System of Higher Education, Pittsburgh, PA (US)

(72) Inventors: Jennifer M. Bomberger, Orford, NH (US); Ananya Kapur, Kennett Square, PA (US)

(21) Appl. No.: 18/495,903

(22) Filed: Oct. 27, 2023

Related U.S. Application Data
(60) Provisional application No. 63/381,154, filed on Oct. 27, 2022.

(51) Int. Cl. C12N 1/20; C12N 5/00

(52) U.S. Cl. CPC C12N 1/20; C12N 5/0018; C12R 2001/445

(57) ABSTRACT

An airway model for culturing microbes such as bacteria Pseudomonas aeruginosa, is provided, comprising culturing the microbes in synthetic sputum media on an air-liquid interface culture of airway epithelial cells. The bacteria may be Pseudomonas aeruginosa, and the epithelial cells may be from a cystic fibrosis (CF) patient and/or comprise a mutation in CFTR found in CF patients.

### CROSS REFERENCE TO RELATED APPLICATIONS

This application claims priority to U.S. Provisional Patent Application No. 63/381,154 filed Oct. 27, 2022, the disclosure of which is incorporated herein by reference in its entirety.

Research for this invention was supported by awards from the Cystic Fibrosis Foundation.

Small animal models currently do not accurately replicate the infection environment that exists during chronic bacterial infections of the respiratory tract. The development of new antimicrobial approaches has been hindered by the inability to recapitulate the human airways for the study of chronic bacterial infections, particularly those involving the development of bacterial biofilms. For example, the opportunistic pathogen Pseudomonas aeruginosa and Staphylococcus aureus are common causes of chronic cystic fibrosis (CF) lung infection. Other common opportunistic pathogens in CF patients include Burkholderia spp., Achromobacter spp., Stenotrophomonas maltophilia, anaerobes, nontuberculous mycobacteria, and fungi. Previously, biofilms have been prepared, and artificial sputum has been utilized to culture bacteria. Aiyer A, et al. describes the makeup and evolution of artificial sputum (Aiyer A, Manos J., The Use of Artificial Sputum Media to Enhance Investigation and Subsequent Treatment of Cystic Fibrosis Bacterial Infections. Microorganisms. 2022 Jun. 22; 10(7):1269, see also, e.g., Neve R L, Carrillo B D, Phelan V V. Impact of Artificial Sputum Medium Formulation on Pseudomonas aeruginosa Secondary Metabolite Production. J Bacteriol. 2021 Oct. 12; 203(21):e0025021, describing multiple non-limiting examples of artificial sputum medium, including SCFM2 among many others), including synthetic sputum medium as tested in the examples below (SCFM2). While artificial sputum such as SCFM2 can elicit expression of a variety of P. aeruginosa genes, it is not sufficiently accurate in its ability to mimic natural gene expression in CF patient's sputum, as it does not contain the respiratory epithelium, as is part of the infection environment in a patient. As such, further advances are needed to provide an artificial system for evaluating therapeutics for treatment of airway infections, such as P. aeruginosa infections in CF patients.

### SUMMARY

According to a first aspect, a method of modeling a microbe biofilm is provided, comprising: culturing a bacterium or a sputum sample comprising a microbe in a first synthetic sputum media to produce a microbe culture; and depositing and culturing the microbe culture in a second synthetic sputum media onto an air liquid interface culture of differentiated airway epithelial cells.

According to a second aspect, a cell culture device is provided comprising an air-liquid interface culture of differentiated airway epithelial cells and synthetic sputum medium comprising a bacteria or sputum sample deposited on the airway epithelial cells.

According to a third aspect, a kit is provided comprising an air liquid interface culture of airway epithelial cells, e.g., bronchial cells, and synthetic sputum medium in a container separate from the cell culture device.

The following numbered clauses outline various aspects, embodiments, or examples of the present invention.

Clause 1. A method of modeling a microbe biofilm, comprising: culturing a bacterium or a sputum sample comprising a microbe in a first synthetic sputum media to produce a microbe culture; and depositing and culturing the microbe culture in a second synthetic sputum media onto an air liquid interface culture of differentiated airway epithelial cells.

Clause 2. The method of clause 1, wherein the airway epithelial cells are bronchial epithelial cells or immortalized bronchial epithelial cells.

Clause 3. The method of clause 1 or 2, wherein the airway epithelial cells are human.

Clause 4. The method of any one of clauses 1-3, wherein the airway epithelial cells comprise a CFTR-associated mutation.

Clause 5. The method of clause 4, wherein the CFTR mutation is a ΔF508 CFTR mutation.

Clause 6. The method of clause 1, wherein the airway epithelial cells comprise CFBE41o-, 16HBE, Calu3, CFT-1, or JME cells.

Clause 7. The method of any one of clauses 1-6, wherein the microbe is a bacteria.

Clause 8. The method of clause 7, wherein the bacteria is selected from Pseudomonas aeruginosa, Staphylococcus aureus (e.g., Methicillin-resistant Staphylococcus aureus (MRSA)), Mycobacteria spp., Burkholderia spp., Achromobacter spp., or Stenotrophomonas maltophilia.

Clause 9. The method of clause 8, wherein the microbe is Pseudomonas aeruginosa.

Clause 10. The method of any one of clauses 1-9, wherein the microbe is obtained from a sputum sample from a patient.

Clause 11. The method of any one of clauses 1-10, wherein the second synthetic sputum medium comprises SCFM2 synthetic sputum medium.

Clause 12. The method of any one of clauses 1-11, wherein the first synthetic sputum medium comprises SCFM synthetic sputum medium.

Clause 13. The method of any one of clauses 1-12, comprising culturing the microbe culture in synthetic sputum media on the airway epithelial cells for from four to 12 hours.

Clause 14. The method of clause 1, comprising:
preparing an air-liquid interface culture of differentiated airway epithelial cells;
obtaining a microbe sample and mixing the microbe sample with a first synthetic sputum media, to form a first culture and culturing the first culture for from 4 to 48 hours;
mixing an aliquot, or a diluted (e.g., a 10², 10³, 10⁴, 10⁵, 10⁶, 10⁷, and/or 10⁸ dilution) aliquot, of the cultured first culture with a second synthetic sputum media, to form a second culture and culturing the second culture for from 4 to 48 hours;
depositing an aliquot, or a diluted aliquot (e.g., a 10², 10³, 10⁴, 10⁵, 10⁶, 10⁷, and/or 10⁸ dilution) in the second synthetic sputum media, of the cultured second culture in the second synthetic sputum media, onto the airway side of the epithelial cells in the air liquid interface culture and culturing for from four to 24 hours, optionally in the presence of an antimicrobial or antibiotic agent in the second synthetic sputum media or cell culture media or the air-liquid interface culture; and
removing a sample of the second synthetic sputum media including the microbe cells, and quantifying the number of cells in the sample.

Clause 15. The method of clause 1, comprising:
preparing an air-liquid interface culture of differentiated CF bronchial epithelial cells;
obtaining a bacteria sample, such as a sputum sample from a CF patient or a culture of a bacteria, and mixing the bacteria sample with an SCFM synthetic sputum media to form a first culture and culturing while shaking at 37° C. for about 16 hours;
mixing an aliquot, or a diluted (e.g., a 10², 10³, 10⁴, 10⁵, 10⁶, 10⁷, and/or 10⁸ dilution) aliquot, of the cultured first culture with SCFM2 synthetic sputum media to form a second culture and culturing while shaking at 37° C. for about 16 hours;
depositing an aliquot, or a diluted aliquot (e.g., a 10², 10³, 10⁴, 10⁵, 10⁶, 10⁷, and/or 10⁸ dilution) in the SCFM2 synthetic sputum media, of the cultured second culture in the SCFM2 synthetic sputum media onto the airway side of the epithelial cells in the air-liquid interface culture and culturing for about 8 hours in an incubator at 37° C., optionally in the presence of an antimicrobial or antibiotic agent in the SCFM2 or cell culture media or the air-liquid interface culture; and
removing a sample of the SCFM2 synthetic sputum media including the bacteria cells, and quantifying the number of bacteria cells in the sample.

Clause 16. The method of clause 15, wherein the bacteria is Pseudomonas aeruginosa.

Clause 17. The method of clause 1, further comprising adding an antimicrobial composition, such as an antibiotic, to the air-liquid interface culture and quantifying and/or qualitatively evaluate growth of the microbe in the air-liquid interface culture to determine efficacy, e.g., a minimum inhibitory concentration or other measure of antimicrobial efficacy, of the antimicrobial composition against the bacteria.

Clause 18. The method of any one of clauses 1-17, for use in modeling an airway disease, such as chronic airway infection, cystic fibrosis, chronic obstructive pulmonary disease, and/or bronchiectasis.

Clause 19. A cell culture device comprising an air-liquid interface culture of differentiated airway epithelial cells and synthetic sputum medium comprising a bacteria or sputum sample deposited on the airway epithelial cells.

Clause 20. The device of clause 19, wherein the airway epithelial cells are human.

Clause 21. The device of clause 19 or 20, wherein the airway epithelial cells comprise a CFTR-associated mutation

Clause 22. The device of clause 21, wherein the airway epithelial cells are CFBE41o-, 16HBE, Calu3, CFT-1, or JME cells or do not express CFTR, e.g., comprise a ΔF508 CFTR mutation.

Clause 23. The device of any one of clauses 18-22, wherein the synthetic sputum medium comprises SCFM2 synthetic sputum medium.

Clause 24. A kit comprising an air liquid interface culture of airway epithelial cells, e.g., bronchial cells, and synthetic sputum medium in a container separate from the cell culture device.

Clause 25. The kit of clause 24, wherein the airway epithelial cells are human.

Clause 26. The kit of clause 24 or 25, wherein the airway epithelial cells comprise a CFTR-associated mutation.

Clause 27. The kit of clause 26, wherein the airway epithelial cells are CFBE41o-, 16HBE, Calu3, CFT-1, or JME cells or do not express CFTR, e.g., comprise a ΔF508 CFTR mutation.

Clause 28. The kit of any one of claims 24-27, wherein the synthetic sputum medium comprises SCFM2 synthetic sputum medium.

### BRIEF DESCRIPTION OF THE DRAWINGS

FIG. 1 provides a schematic of a culture method as described herein.

FIG. 2 is a table providing an exemplary composition of SCFM2.

FIGS. 3A and 3B are graphs showing (FIG. 3A), SCFM2 and P. aeruginosa biofilms do not disrupt CF airway epithelial cell integrity as shown by transepithelial electrical resistance (TEER) measurements, and (FIG. 3B) P. aeruginosa reproducibly forms biofilms in luminal space above epithelial cells in SCFM2 and associated with the CF epithelial cell surface.

FIGS. 4A and 4B are graphs showing (FIG. 4A), SCFM2 and clinically-isolated P. aeruginosa biofilms do not disrupt CF airway epithelial cell integrity as shown by transepithelial electrical resistance (TEER) measurements, and (FIG. 4B) clinically-isolated P. aeruginosa reproducibly forms biofilms in luminal space above epithelial cells in SCFM2 and associated with the CF epithelial cell surface.

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of ranges is intended as a continuous range including every value between the minimum and maximum values. As used herein “a” and “an” refer to one or more.

As used herein, the term “comprising” is open-ended and may be synonymous with “including”, “containing”, or “characterized by”. The term “consisting essentially of” limits the scope of a claim to the specified materials or steps and those that do not materially affect basic and novel characteristic(s). The term “consisting of” excludes any element, step, or ingredient not specified in the claim. As used herein, embodiments “comprising” one or more stated elements or steps also include but are not limited to embodiments “consisting essentially of” and “consisting of” these stated elements or steps.

As used herein, the term “patient” or “subject” refers to members of the animal kingdom including but not limited to human beings and “mammal” refers to all mammals, including, but not limited to human beings.

A model system, including methods, devices, and kits, is provided that reflects the mucosal environment of the human respiratory tract, combining a synthetic airway surface media that mimics sputum and culture of air-liquid interface differentiated human bronchial epithelial cells, to which bacterial aggregates are inoculated. The resulting microbial infection model captures the bacterial biofilms growing in the respiratory tract during chronic infections, as assessed by transcriptomics and imaging analyses. The model is further adapted to support use of human bronchial epithelial cell lines, as well as well-differentiated primary human bronchial epithelial cells, and lab strains of Pseudomonas aeruginosa, as well as clinical isolates from respiratory infections. This model has applications in screening for new antimicrobials, as well as use for host-pathogen interactions research.

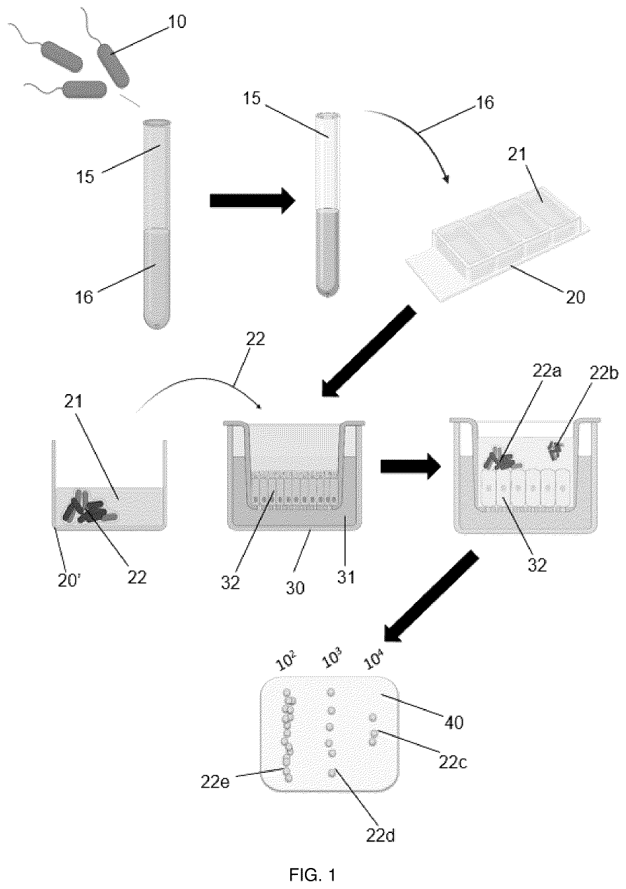

Methods of culturing microbes are provided herein. Referring to FIG. 1, microbes 10, such as bacteria, and in one example P. aeruginosa, are inoculated into a vessel 15, such as a tube, comprising a first synthetic sputum media 16, such as SCFM as an example. Other synthetic sputum media may be utilized. Microbes 10 are incubated in the synthetic sputum media 16 for a suitable time period, such as, for example and without limitation, from 8 to 48 hours at 37° C., and in one example microbes 10 are incubated in SCFM for 16 hours at 37° C. After this primary incubation step, microbe cells 22 from the synthetic medium are transferred into a second vessel 20 comprising a second synthetic sputum media 21 that may be the same or different than the first synthetic sputum media 16, and may be SCFM2, for example and without limitation, and may be cultured for from 0 to 24 hours at 37° C. Culture temperatures and conditions may be varied. In one example, the microbe cells 22 are incubated in SCFM2 for 16 hours at 37° C. Examples of specific synthetic sputum media. Including SCFM, SCFM2, SCFM3, etc., are broadly-known, as indicated in Aiyer A, et al. Microorganisms. 2022 Jun. 22; 10(7):1269 and Neve R L, et al. J Bacteriol. 203(21):e0025021.

Once incubated for a sufficient time microbe cells 22 in the synthetic sputum media 21 are removed from vessel 20′ (depicting one well from multi-well vessel 20), and may be diluted in the same or different synthetic sputum media, and are transferred to an air-facing side of an air-liquid interface vessel 30 comprising media in the basolateral compartment 31 and differentiated airway epithelial cells 32. Differentiated epithelial cells 32 may be cultured in any effective manner, such as, for example, as described in Fulcher et al. (Fulcher M L, et al. Well-differentiated human airway epithelial cell cultures. Methods Mol Med. 2005; 107:183-206) and Jiang D, et al. (Jiang D, et al. Air-Liquid Interface Culture of Human and Mouse Airway Epithelial Cells. Methods Mol Biol. 2018; 1809:91-109). Basolateral media 31 may be any suitable airway epithelial cell growth media, such as Airway Epithelial Cell Growth Medium (Sigma-Aldrich) or BEBM™ Bronchial Epithelial Cell Growth Basal Medium (Lonza). The airway epithelial cells 32 are grown in the air-liquid interface culture 30 for at least 7 days and up to 28 days, then inoculated with SCFM2-grown bacterial aggregates for suitable time periods, from four to 48 hours, e.g. 8 hours at 37° C. In examples, two types of biofilms may be formed, namely SCFM2-epithelial biofilms associated with the epithelial surface 22a and in the SCFM2 media above the epithelial surface 22b. Biomolecules, such as RNA or protein may be extracted from microbe cells 22 of biofilms 22a and/or 22b to evaluate microbe growth and gene expression. Microbe cells 22 of biofilms 22a and/or 22b, may be plated on suitable microbe growth media 40, in various dilutions, such as 10⁴ (22c), 10³ (22d), and 10³ (22e), in suitable media to evaluate bacterial growth.

In use, the method depicted in FIG. 1, may be used to test antibiotic or antimicrobial compounds for their activity on the microbe cells 22. For example, multiple air-liquid interface cultures with the microbe cells 22 may be prepared, and incubated with various dilutions of the compounds to be tested in either the media 31 and/or synthetic sputum media 21, with control compound(s) tested as positive control(s), or with no compound tested as a negative control. The microbes 10 may be a bacteria, such as Pseudomonas aeruginosa, Staphylococcus aureus (e.g., Methicillin-resistant Staphylococcus aureus (MRSA)), Mycobacteria spp., Burkholderia spp., Achromobacter spp., or Stenotrophomonas maltophilia. Likewise sensitivity of other bacteria of fungi to antibiotic or antimicrobial compounds, or compounds in combinations, may be tested as described.

As a non-limiting example, a culture of sputum cells may be prepared by:
preparing an air-liquid interface differentiated culture of CF bronchial epithelial cells;
obtaining a microbe sample, such as a sputum sample from a CF patient or a culture of a microbe, such as a commercially-available microbe sample, e.g., from the ATCC (American Type Culture Collection) or other depository;
mixing the microbe or sputum with a first synthetic sputum media, such as SCFM, to form a first culture and culturing while shaking at 37° C. for 16 hours;
mixing an aliquot, or a diluted (e.g., a 10², 10³, 10⁴, 10⁵, 10⁶, 10⁷, and/or 10⁸ dilution) aliquot, of the cultured first culture with a second synthetic sputum media, such as SCFM2, to form a second culture and culturing while shaking at 37° C. for 16 hours;
depositing an aliquot, or a diluted aliquot (e.g., a 10², 10³, 10⁴, 10⁵, 10⁶, 10⁷, and/or 10⁸ dilution) in the second synthetic sputum media, such as SCFM2, of the cultured second culture in the second synthetic sputum media, such as SCFM2, onto the airway side of the epithelial cells in the air liquid interface culture and culturing for from four to 24 hours, e.g., for 8 hours in an incubator at 37° C., e.g., with 5% CO₂, optionally in the presence of an antimicrobial or antibiotic agent; and
removing a sample of the second synthetic sputum media, such as SCFM2, including the cells, and quantifying the number of cells in the sample, e.g., by plating of dilutions of the cells.

Cells useful in the described methods and devices may be airway epithelial cells, which may be human airway epithelial cells. In one example, the airway epithelial cells are bronchial epithelial cells, such as human bronchial epithelial cells. Airway epithelial cells in all instances may be a primary culture, or a differentiated culture, differentiated from progenitor cells, such as stem cells. The airway epithelial cells may be a cell line (immortalized cells) such as 16HBE (e.g., 16HBE14o-Human Bronchial Epithelial Cell Line, Sigma-Aldrich), Calu3 (HTB-55™, ATCC), CFT-1 (see, e.g., Vázquez E, et al. Defective regulatory volume decrease in human cystic fibrosis tracheal cells because of altered regulation of intermediate conductance Ca2+-dependent potassium channels. Proc Natl Acad Sci USA. 2001 Apr. 24; 98(9):5329-34), JME (e.g., JME/CF15, see, e.g., Tong Z, et al. Prostasin, a membrane-anchored serine peptidase, regulates sodium currents in JME/CF15 cells, a cystic fibrosis airway epithelial cell line. Am J Physiol Lung Cell Mol Physiol. 2004 November; 287(5):L928-35), or CFBE41o- (Human C F Bronchial Epithelial Cell Line, Sigma-Aldrich, and Ehrhardt C, Collnot E M, Baldes C, Becker U, Laue M, Kim K J, Lehr C M. Towards an in vitro model of cystic fibrosis small airway epithelium: characterisation of the human bronchial epithelial cell line CFBE41o-. Cell Tissue Res. 2006 March; 323(3):405-15, and see, generally, Gruenert D C, et al. Established cell lines used in cystic fibrosis research. J Cyst Fibros. 2004 August; 3 Suppl 2:191-6.). Primary human bronchial epithelium from patients may be utilized (see, e.g., Neuberger T, Burton B, Clark H. Van Goor F. Use of primary cultures of human bronchial epithelial cells isolated from cystic fibrosis patients for the pre-clinical testing of CFTR modulators. Methods Mol Biol. 2011; 741:39-54), e.g., differentiated at the air-liquid interface. Airway epithelial cells may be human cells that include or are modified to include a CFTR mutation associated with cystic fibrosis, such as the Δ508 mutation, or other mutations (See, e.g., Bobadilla J L, Macek M Jr, Fine J P, Farrell P M. Cystic fibrosis: a worldwide analysis of CFTR mutations—correlation with incidence data and application to screening. Hum Mutat. 2002 June; 19(6):575-606, for example Table 1 thereof). Cells may be primary, obtained from OF patients, or immortalized versions thereof.

An air-liquid interface airway epithelial cell culture is a culture of airway epithelial cells on a permeable membrane in contact with culture medium in a culture vessel, and having a media-facing (e.g., permeable membrane-facing) side, and an air-facing side opposite the liquid-facing side onto which synthetic sputum may be deposited (See, air-liquid interface vessel 30, epithelial cells 32, and synthetic sputum media 21 depicted in FIG. 1, as a schematic example). The epithelial cell culture may be a differentiated layer of epithelial cells. Suitable permeable membranes, such as inserts are broadly-available, such as Transwell®, Snapwell™, Netwell®, and Falcon® permeable supports (Corning) among other commercially-available permeable supports. See, air-liquid interface vessel 30 depicted in FIG. 1, as a schematic example.

Microbes include any eukaryotic, prokaryotic, or viral organism, which may be pathogenic in humans and in human cystic fibrosis patients. In one example, the microbes are bacterial, and in another example, the microbes are fungal, such as, for example and without limitation, Candida spp. and Aspergillus spp. Specific examples of microbes include, without limitation: Pseudomonas aeruginosa, Staphylococcus aureus (e.g., Methicillin-resistant Staphylococcus aureus (MRSA)), Mycobacteria spp., Burkholderia spp., Achromobacter spp., or Stenotrophomonas maltophilia. In use in the described assay, the microbe may be present in sputum of a patient, or may be a reference strain of the microbe, such as, for example and without limitation, the MPAO1 strain of P. aeruginosa.

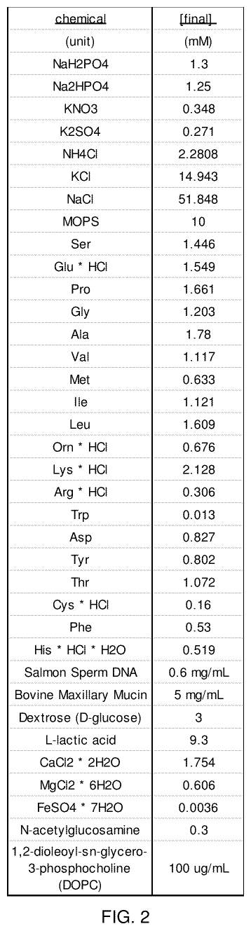

Synthetic sputum media may be any artificial sputum, for example and without limitation, as are broadly-known. As above, Aiyer, et al. and Neve, et al. describe the makeup and evolution of artificial sputum (Aiyer A, et al. Microorganisms. 2022 Jun. 22; 10(7):1269 and Neve R L, et al. J Bacteriol. 2021 Oct. 12; 203(21):e0025021), including synthetic sputum media as tested in the examples below (SCFM and SCFM2). Turner et al. (Turner K H, Wessel A K, Palmer G C, Murray J L, Whiteley M. Essential genome of Pseudomonas aeruginosa in cystic fibrosis sputum. Proc Natl Acad Sci USA. 2015 Mar. 31; 112(13):4110-5) provides a recipe for SCM2 and, with Aiyer, et al. and Neve, et al. describe the differences between SCFM2 and SCFM (SCFM1, see, e.g., Table 1 of Palmer et al. Nutritional cues control Pseudomonas aeruginosa multicellular behavior in cystic fibrosis sputum. J Bacteriol. 2007 November; 189(22):8079-87), such as the addition of increased amounts of DNA, mucin, and FeSO₄, and including DOPC (e.g., 1,2-Dioleoyl-sn-glycero-3-phosphocholine) and various amino acids. FIG. 2 provides an exemplary composition of SCM2.

### EXAMPLES

Models currently exist for synthetic sputum media and models that culture bacterial biofilms with human bronchial epithelial cells. The model described herein combines those features through extensive trial and error to develop a first of its kind, combined synthetic sputum and epithelial co-culture bacterial biofilm model. Bacterial aggregates may be first grown in less-complex synthetic sputum before inoculating and growing these aggregates to form biofilms with human bronchial epithelial cells. As there are no existing bacterial biofilm models for the respiratory tract that closely mimic the human chronic infections, this model is a major step forward in modeling the human airways in a tractable way while studying bacterial biofilms. Screening platforms for new antimicrobials are of great interest to the biotech, pharmaceutical and FDA, which this new model offers.

### Example 1

Mutations in CFTR chloride channel cause thick mucus to line the airway, promoting chronic Pseudomonas aeruginosa infections for patients with cystic fibrosis (CF). There are a paucity of animal and in vitro models that accurately mimic host-pathogen interactions in the CF respiratory tract. In a previous study, a computational approach was established to evaluate the accuracy of different in vitro models to replicate P. aeruginosa expression in CF sputum.

Accuracy scores were assigned to each model based on their ability to recapitulate gene expression of P. aeruginosa in CF sputum (Cornforth, D. M., Diggle, F. L., Melvin, J. A., Bomberger, J. M., & Whiteley, M. (2020). Quantitative framework for model evaluation in microbiology research using Pseudomonas aeruginosa and cystic fibrosis infection as a test case. MBio, 11(1), e03042-19). It was found that synthetic cystic fibrosis media (SCFM2) and the “original” bacterial biofilm with epithelial coculture model had the highest accuracy scores compared to all other models at 86% and 84%, respectively. SCFM2 and the “original” bacterial biofilm with epithelial coculture missed 783 and 896 genes, respectively, compared to CF sputum, but when computationally combined, they only missed 363 genes. It is proposed that by integrating SCFM2 and epithelial coculture models, we can improve the accuracy scores, thus providing a more accurate representation of P. aeruginosa physiology in CF sputum (see, e.g., Lewin G R, et al. Application of a quantitative framework to improve the accuracy of a bacterial infection model. Proc Natl Acad Sci USA. 2023 May 9; 120(19):e2221542120).

### Example 2—Pseudomonas aeruginosa Forms Biofilms in Combined SCFM2 Airway Epithelial Model

Initial experiments establish that P. aeruginosa reproducibly forms biofilms on CF epithelial cell air-liquid interface cultures. MPAO1 strain P. aeruginosa was cultured in SCFM overnight, then inoculated in SCFM2 statically. After ˜16 hours, the cells were then subcultured to the airway luminal side of air-liquid interface differentiated CF bronchial epithelial cells (CFBE41o-) for 8 hours.

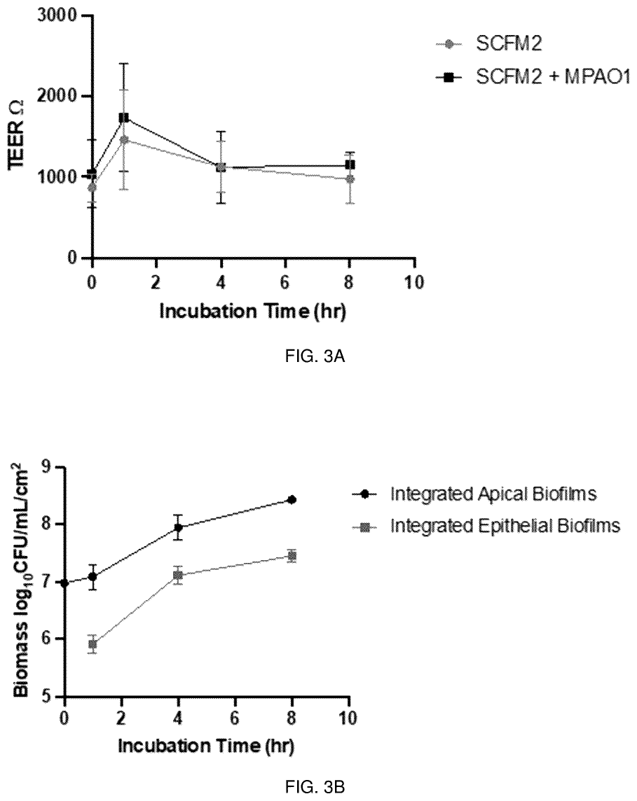

As shown in FIG. 3A, epithelial cell integrity is not disrupted by microbial biofilm production. Specifically, SCFM2 and Pseudomonas aeruginosa (MPAO1 strain) biofilms do not disrupt CF airway epithelial cell (CFBE41o-) integrity as shown by transepithelial electrical resistance (TEER) measurements. Further, as shown in FIG. 3B, Pseudomonas aeruginosa (MPAO1) reproducibly forms biofilms in luminal space above epithelial cells in SCFM2 and associated with the CF epithelial cell surface, with a time dependent increase in bacterial counts in both the apical supernatant and associated with the respiratory epithelium, peaking at 8 hours post-infection. Confocal imaging at 1, 4, and 8 hours post-inoculation (data not shown, but provided in priority application, U.S. Provisional Patent Application No. 63/381,154 filed Oct. 27, 2022) show that single bacteria and small bacterial aggregates first associate with the epithelial cell surface. These aggregates grow over the incubation period similar to the size and morphology of those observed in the CF lung.

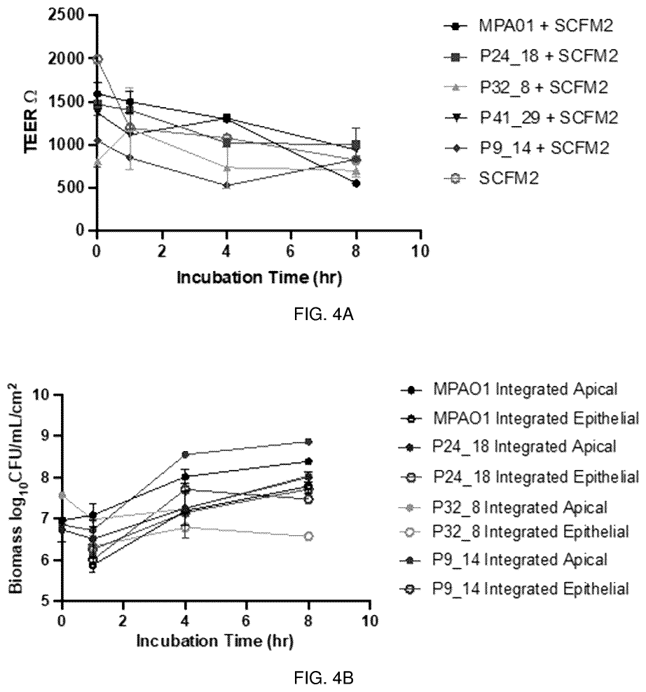

The above experiments were repeated using clinical P. aeruginosa isolates. In brief, CF clinical isolates of P. aeruginosa were cultured in SCFM overnight, then inoculated in SCFM2 statically. After ˜16 hours, the cells were then subcultured to the airway luminal side of air-liquid interface differentiated CF bronchial epithelial cells (CFBE41o-) for 8 hours. Results show (FIG. 4A) that the clinical isolates do not affect epithelial cell integrity, and (FIG. 4B) reproducible formation of biofilms in the luminal space above the epithelial cells.

The present invention has been described with reference to certain exemplary embodiments, dispersible compositions and uses thereof. However, it will be recognized by those of ordinary skill in the art that various substitutions, modifications or combinations of any of the exemplary embodiments may be made without departing from the spirit and scope of the invention. Thus, the invention is not limited by the description of the exemplary embodiments, but rather by the appended claims as originally filed.

1. A method of modeling a microbe biofilm, comprising: culturing a bacterium or a sputum sample comprising a microbe in a first synthetic sputum media to produce a microbe culture; and depositing and culturing the microbe culture in a second synthetic sputum media onto an air liquid interface culture of differentiated airway epithelial cells.

2. The method of claim 1, wherein the airway epithelial cells are bronchial epithelial cells or immortalized bronchial epithelial cells.

3. The method of claim 1, wherein the airway epithelial cells are human.

4. The method of claim 1, wherein the airway epithelial cells comprise a CFTR-associated mutation.

5. The method of claim 4, wherein the CFTR mutation is a ΔF508 CFTR mutation.

6. The method of claim 1, wherein the microbe is a bacteria selected from Pseudomonas aeruginosa, Staphylococcus aureus (e.g., Methicillin-resistant Staphylococcus aureus (MRSA)), Mycobacteria spp., Burkholderia spp., Achromobacter spp., or Stenotrophomonas maltophilia.

7. The method of claim 1, wherein the microbe is obtained from a sputum sample from a patient.

8. The method of claim 1, wherein the second synthetic sputum medium comprises SCFM2 synthetic sputum medium and/or the first synthetic sputum medium comprises SCFM synthetic sputum medium.

9. The method of claim 1, comprising culturing the microbe culture in synthetic sputum media on the airway epithelial cells for from four to 12 hours.

10. The method of claim 1, comprising:
preparing an air-liquid interface culture of differentiated airway epithelial cells;
obtaining a microbe sample and mixing the microbe sample with a first synthetic sputum media, to form a first culture and culturing the first culture for from 4 to 48 hours;
mixing an aliquot, or a diluted aliquot, of the cultured first culture with a second synthetic sputum media, to form a second culture and culturing the second culture for from 4 to 48 hours;
depositing an aliquot, or a diluted aliquot in the second synthetic sputum media, of the cultured second culture in the second synthetic sputum media, onto the airway side of the epithelial cells in the air liquid interface culture and culturing for from four to 24 hours, optionally in the presence of an antimicrobial or antibiotic agent in the second synthetic sputum media or cell culture media or the air-liquid interface culture; and
removing a sample of the second synthetic sputum media including the microbe cells, and quantifying the number of cells in the sample.

11. The method of claim 1, comprising:
preparing an air-liquid interface culture of differentiated CF bronchial epithelial cells;
obtaining a bacteria sample, such as a sputum sample from a CF patient or a culture of a bacteria, and mixing the bacteria sample with an SCFM synthetic sputum media to form a first culture and culturing while shaking at 37° C. for about 16 hours;
mixing an aliquot, or a diluted aliquot, of the cultured first culture with SCFM2 synthetic sputum media to form a second culture and culturing while shaking at 37° C. for about 16 hours;
depositing an aliquot, or a diluted aliquot in the SCFM2 synthetic sputum media, of the cultured second culture in the SCFM2 synthetic sputum media onto the airway side of the epithelial cells in the air-liquid interface culture and culturing for about 8 hours in an incubator at 37° C., optionally in the presence of an antimicrobial or antibiotic agent in the SCFM2 or cell culture media or the air-liquid interface culture; and
removing a sample of the SCFM2 synthetic sputum media including the bacteria cells, and quantifying the number of bacteria cells in the sample.

12. The method of claim 1, further comprising adding an antimicrobial composition, such as an antibiotic, to the air-liquid interface culture and quantifying and/or qualitatively evaluate growth of the microbe in the air-liquid interface culture to determine efficacy of the antimicrobial composition against the bacteria.

13. A cell culture device comprising an air-liquid interface culture of differentiated airway epithelial cells and synthetic sputum medium comprising a bacteria or sputum sample deposited on the airway epithelial cells.

14. The device of claim 13, wherein the airway epithelial cells are human.

15. The device of claim 13, wherein the airway epithelial cells comprise a CFTR-associated mutation.

16. The device of claim 15, wherein the airway epithelial cells are CFBE41o-, 16HBE, Calu3, CFT-1, or JME cells or do not express CFTR, e.g., comprise a ΔF508 CFTR mutation.

17. The device of claim 13, wherein the synthetic sputum medium comprises SCFM2 synthetic sputum medium.

18. A kit comprising an air liquid interface culture of airway epithelial cells and synthetic sputum medium in a container separate from the cell culture device.

19. The kit of claim 18, wherein the airway epithelial cells are human.

20. The kit of claim 18, wherein the airway epithelial cells comprise a CFTR-associated mutation, wherein the airway epithelial cells are CFBE41o-, 16HBE, Calu3, CFT-1, or JME cells or do not express CFTR, e.g., comprise a ΔF508 CFTR mutation, and/or wherein the synthetic sputum medium comprises SCFM2 synthetic sputum medium.
